(19) United States
(12) Patent Application Publication
(10) Pub. No.: US 2011/0160841 A1
(43) Pub. Date: Jun. 30, 2011

(54) MEDICAL APPLIANCE AND SURFACE TREATMENT METHOD THEREOF

(75) Inventors: Wei-Ching Wang, Kaohsiung County (TW); Wei-Jen Shih, Kaohsiung County (TW); Jin-Long Jou, Kaohsiung City (TW)

(73) Assignee: METAL INDUSTRIES RESEARCH&DEVELOPMENT CENTRE, KAOHSIUNG (TW)

(21) Appl. No.: 12/964,416

(22) Filed: Dec. 9, 2010

(30) Foreign Application Priority Data
Dec. 29, 2009 (TW) 098145500

(51) Int. Cl. A61F 2/82; C25D 5/16; C25D 11/02; A61B 17/68; A61B 17/80; A61M 25/09; A61B 17/00

(52) U.S. Cl. 623/1.15; 205/188; 205/171; 606/301; 606/70; 604/528; 606/1

(57) ABSTRACT

A surface treatment method for a medical appliance is provided. The surface treatment method includes providing a metal layer; forming an intermediate layer on a surface of the metal layer, in which a thickness of the intermediate layer is greater than a thickness of a native oxide layer of the metal surface; and grafting a functional polymer on the intermediate layer through an electrodeposition process.

### CROSS-REFERENCE TO RELATED APPLICATION

This application claims the benefit of Taiwan Patent Application No. 098145500, filed on Dec. 29, 2009, which is hereby incorporated by reference for all purposes as if fully set forth herein.

### BACKGROUND OF THE INVENTION

1. Field of Invention

The present invention relates to a surface treatment method of metal and a medical appliance, and more particularly to a treatment method for grafting a functional polymer on a surface of metal.

2. Related Art

Medical appliances that can be implanted in human bodies, such as bone screws, bone plates, and cardiac stents, are usually used to fix or support bones or blood vessels in human bodies. Or, medical appliances that can be inserted in human bodies temporarily such as metal guide wire and surgical instruments usually have functions of surgical guidance or operations. Medical appliances that can be used outside human bodies such as surgical wrenches and medical metal basins are usually used in surgical processes.

According to the products of the above medical appliances, currently surfaces of the metal guide wires are covered with special functional polymers, for example. In US Patent Publication No. US 20090124984 entitled “MEDICAL APPLIANCE AND PROCESS FOR PRODUCING THE APPLIANCE”, Hanawa discloses a medical appliance with a metal surface, and a hydrophilic organic compound is directly fixed on the metal surface through an electrochemical method. Therefore, a lubrication effect of the surface can be realized through the hydrophilic organic compound.

However, in the contents of the above patent publication, a grafting ratio of the hydrophilic organic compound grafted on the medical appliance is limited by the number of hydroxyl groups (—OH) on the surface of the medical appliance. Therefore, the lubrication effect of the metal surface is influenced by the grafting ratio of the hydrophilic organic compound, so that its effects of effective tissue anti-adhesion and protein anti-adhesion are limited.

### SUMMARY OF THE INVENTION

The present invention is directed to a surface treatment method for a medical appliance. The metal surface treatment method can increase a grafting amount of special functional polymers, thereby further improving effects of tissue anti-adhesion, anti-bacteria, and protein anti-adhesion.

The present invention is further directed to a medical appliance, which can have a grafting amount of special functional polymers, and thus the medical appliance improves effects of tissue anti-adhesion, anti-bacteria, and protein anti-adhesion.

The present invention provides a surface treatment method for a medical appliance, which includes providing a metal layer; forming an intermediate layer on a surface of the metal layer, in which a thickness of the intermediate layer is greater than a thickness of a native oxide layer of the metal surface; and grafting a functional polymer on the intermediate layer through an electrodeposition process.

The present invention further provides a medical appliance, which includes a metal layer, an intermediate layer, and a dissoluble functional polymer. The intermediate layer is formed on the metal layer. A thickness of the intermediate layer is greater than a thickness of a native oxide layer of the surface of the metal layer. The dissoluble functional polymer is grafted on the intermediate layer.

In conclusion, in the present invention, an intermediate layer is formed on the surface of the metal layer through a chemical soak process or an electrochemical anodization process. Different colors are presented according to different thickness of the intermediate layer. Therefore, the products are obviously recognizable. In addition, the formation of the intermediate layer can increase the number of hydroxyl groups (—OH) on the surface of the metal layer.

Additionally, in the present invention, the special functional polymer is then electrochemically grafted on the hydroxyl groups (—OH) of the surface of the intermediate layer (i.e. the oxide layer of the metal layer). The method for forming the intermediate layer can increase the number of hydroxyl groups on the surface effectively, thus this method increases the amount of grafted special functional polymer, so that the effect of tissue anti-adhesion of the product can be improved.

In order to make the above features and advantages of the present invention more comprehensible, the present invention is illustrated below in detail with reference to the embodiments and the accompanying drawings.

### BRIEF DESCRIPTION OF THE DRAWINGS

The present invention will become more fully understood from the detailed description given herein below for illustration only, and thus are not limitative of the present invention, and wherein:

FIG. 1 is a flow chart of a surface treatment method for a medical appliance according to the present invention;

FIGS. 2 to 4 are sectional views of a surface treatment method for a medical appliance according to the present invention;

FIG. 5 shows an anti-tissue adhesion result after titanium metal is implanted under mouse skin for one week;

FIG. 6 shows an anti-tissue adhesion result after titanium metal is implanted under mouse skin for two weeks; and

FIG. 7 shows an anti-tissue adhesion result after titanium metal is implanted under mouse skin for four weeks.

### DETAILED DESCRIPTION OF THE INVENTION

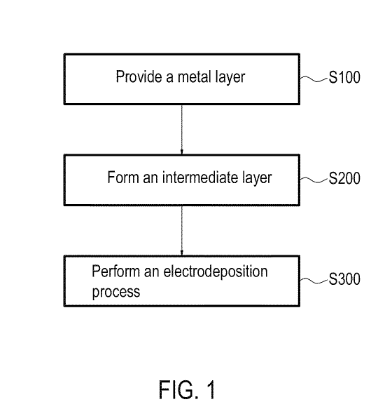

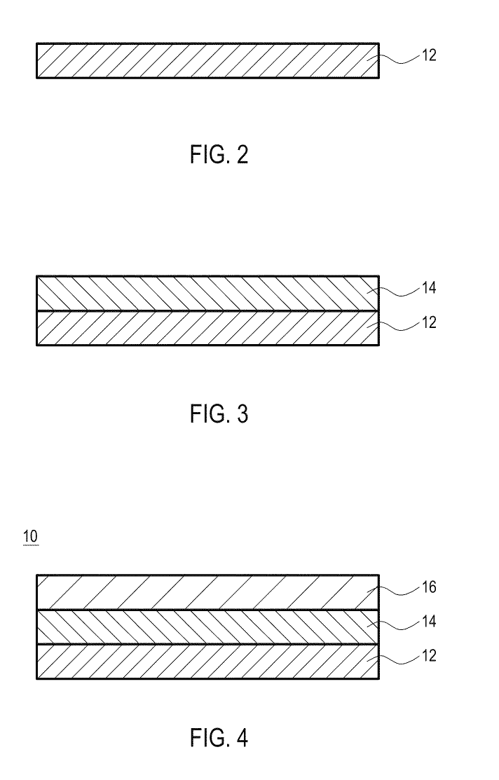

FIG. 1 is a flow chart of a surface treatment method for a medical appliance according to the present invention. FIGS. 2 to 4 are sectional views of a surface treatment method for a medical appliance according to the present invention. Referring to FIGS. 1 to 4, the surface treatment method includes following steps. First, referring to FIG. 2, a metal layer 12 (or a metal body) is provided (Step S100). Material of the metal layer 12 can be titanium metal, alloy containing titanium element, vitallium (Co—Cr—Mo) or stainless steel.

Subsequently, referring to FIG. 3, an intermediate layer 14 is formed on a surface of the metal layer 12 (Step S200). It should be noted that a thickness of the intermediate layer 14 here is greater than a thickness of a native oxide layer of the surface of the metal layer. In particular, for the intermediate layer 14, taking a titanium oxide layer as an example, the thickness of the native oxide layer of the surface of the metal layer 12 is about 10 nanometers (nm) when the surface of the metal layer 12 has the native oxide layer in the prior art. The thickness of the intermediate layer 14 here is between 10 nm and 20 micrometers (μm).

The intermediate layer 14 can be formed on the surface of the metal (Step S200) through a chemical soak process. The chemical soak process includes the following steps. The metal layer 12 is soaked in an acid solution and the acid solution is hydrogen peroxide (H₂O₂) solution or solution that can form a metal oxide layer on the surface of the metal layer 12. For example, after the metal layer 12 is soaked in the acid solution, a titanium oxide layer is formed on the surface of the metal layer 12, and a thickness of the titanium oxide layer is greater than 10 nm.

Alternatively, the intermediate layer 14 is formed on the surface of the metal layer 12 (Step S200) through an electrochemical anodization process. The electrochemical anodization process includes the following steps. The metal layer 12 is placed at an anode and an electrode is placed at a cathode. A current is provided at the metal layer 12. After the electrochemical anodization process, for example a titanium oxide layer is formed on the surface of the metal layer 12, and the thickness of the titanium oxide layer is greater than 10 nm.

Additionally, during the electrochemical anodization process, the thickness of the titanium oxide layer can be controlled by a reaction temperature and a current provided for the reaction. The reaction temperature of the electrochemical anodization process is in a range between −20° C. and 60° C., preferably between −5° C. and 35° C. An externally applied voltage during the electrochemical anodization process can be between 5 volts (V) and 200 V, and preferably between 40 V and 60 V.

Alternatively, the intermediate layer 14 is formed on the surface of the metal layer 12 (Step S200) through a heating process. The heating process includes the following steps. The metal layer 12 is placed in a uniformly heated space and a suitable temperature is provided in a given time. After the heating process, for example a titanium oxide layer is formed on the surface of the metal layer 12, and the thickness of the titanium oxide layer is greater than 10 nm.

Additionally, during the heating process, the thickness of the titanium oxide layer can be controlled by a reaction temperature and the reaction time. The reaction temperature of the heating process is in a range between 100° C. and 1000° C., and preferably between 400° C. and 800° C. The reaction time of the heating process is in a range between 30 minutes and 8 hours, and preferably between 30 minutes and 2 hours.

After the intermediate layer 14 is formed on the surface of the metal layer 12 (Step S200), a functional polymer 16 is grafted on the intermediate layer 14 through an electrodeposition process (Step S300). In particular, the functional polymer 16 is a dissoluble polymer having a terminal amino group, for example, poly ethylene glycol (PEG). The “dissoluble” here means that the functional polymer 16 can be dissolved in water or solvent. The electrodeposition process includes the following steps. The metal layer 12 having the intermediate layer 14 is placed in an electrolyte containing the functional polymer 16, and a current is provided, so as to perform the electrodeposition process.

After the intermediate layer 14 is formed on the surface of the metal layer 12 (Step S200), the medical appliance 10 present different colors (for example, golden, grayish blue, or interlaced gray-blue-green) due to different thickness of the intermediate layer 14, wherein the above colors of the medical appliance 10 are different from a color of the medical appliance 10 having a transparent native oxide layer only (that is, an original color of the metal layer 12). Therefore, the products of the medical appliance 10 are obviously recognizable. In addition, the formation of the intermediate layer 14 can effectively increase the number of hydroxyl groups (—OH) on the surface of the metal layer 12.

In addition, referring to FIG. 4, the functional polymer 16 is grafted on the intermediate layer 14 through an electrodeposition process (Step S300), so as to form the medical appliance 10 according to the present invention. As the number of the hydroxyl groups (—OH) on the surface of the metal layer 12 is effectively increased and the hydroxyl groups (—OH) on the surface can increase grafting positions of the PEG, a PEG grafting ratio can be increased. After the functional polymer 16 is grafted on the surface of the metal layer 12, the effect of tissue anti-adhesion can be improved.

Referring to FIG. 4 again, the medical appliance 10 according to the present invention includes a metal layer 12, an intermediate layer 14, and a dissoluble functional polymer 16. The intermediate layer 14 is formed on the metal layer 12. A thickness of the intermediate layer 14 is greater than a thickness of a native oxide layer of the surface of the metal layer 12. The functional polymer 16 is grafted on the intermediate layer 14. The medical appliance 10 can be an object surgically implanted within bodies, for example, bone screws, bone plates or cardiac stents. Alternatively, the medical appliance 10 can be an object that can be inserted in bodies temporarily, for example, metal guide wires or surgical instruments. Alternatively, the medical appliance 10 can be an object that can be operated outside bodies, for example, surgical wrenches or medical metal basins.

First to third embodiments are used to illustrate the present invention. However, the present invention is not limited to the following embodiments.

### First Embodiment

For a metal implant (that is, a medical appliance) in the first embodiment, titanium metal (that is, the medical appliance 10 includes a metal layer 12 and the metal layer 12 is titanium metal) is taken as an example. It is analyzed that a thickness of the native oxide layer of the surface of the titanium metal is about 10 nm. Then, the titanium metal is soaked in acid solution containing hydrogen peroxide (H₂O₂). The acid solution is 15% H₂O₂. The soak time is 6 hours and the soak temperature is 40° C. The titanium metal is analyzed after soaked in the acid solution. The result shows that the surface of the soaked titanium metal is grayish blue. A thickness of the oxide layer (that is, the intermediate layer 14) on the soaked titanium metal is greater than 300 nm. Finally, an electrodeposition process is performed on the soaked titanium metal, so as to graft the functional polymer 16 (i.e. PEG) on the intermediate layer 14. The functional polymer 16 is transparent, and thus the color of the medical appliance 10 is also grayish blue.

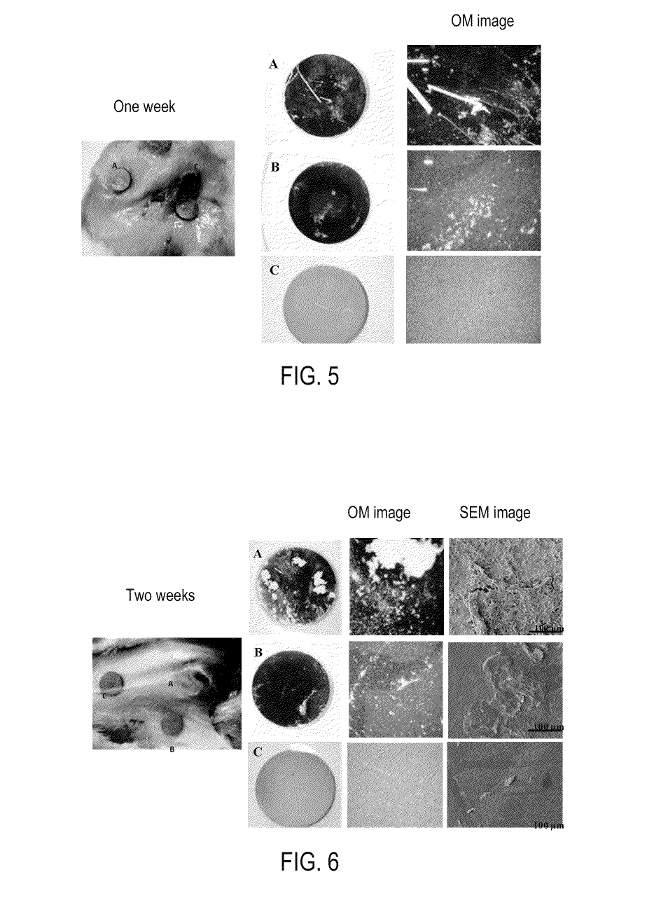

Subsequently, an animal experiment is performed to observe whether the surface treatment method according to the present invention can improve an effect of tissue anti-adhesion. In the animal experiment, (i) the medical appliance (PEG_Ti_H₂O₂): the titanium metal soaked in the acid solution and then grafted with the functional polymer 16 in the first embodiment, (ii) the medical appliance (PEG_Ti): titanium metal directly grafted with the functional polymer 16, and (iii) the medical appliance (CP_Ti): the titanium metal are respectively implanted under the mouse skin to observe the effect of tissue anti-adhesion after implantation for one week, two weeks, and four weeks. The results are as shown in FIGS. 5 to 7.

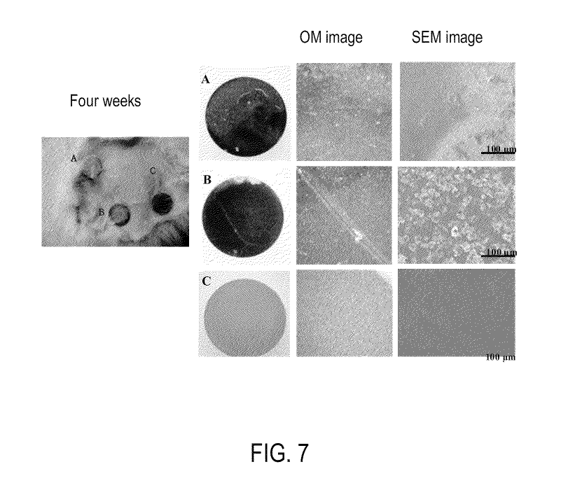

FIG. 5 shows a result of tissue anti-adhesion after the medical appliances are implanted under mouse skin for one week. FIG. 6 shows a result of tissue anti-adhesion after the medical appliances are implanted under mouse skin for two weeks. FIG. 7 shows a result of tissue anti-adhesion after the medical appliances are implanted under mouse skin four weeks. In the FIGs, Group A is a result of tissue anti-adhesion that the medical appliance (CP_Ti): the titanium metal having the native oxide layer is implanted under mouse skin. Group B is a result of tissue anti-adhesion that the medical appliance (PEG_Ti): the titanium metal having the native oxide layer directly grafted with the functional polymer 16 is implanted under mouse skin. Group C is a result of tissue anti-adhesion that the medical appliance (PEG_Ti_H₂O₂): the titanium metal soaked in the acid solution and then grafted with the functional polymer 16 is implanted under mouse skin.

As can be seen through optical microscope (OM) observation of animal experiment results in FIGS. 5 to 7, white areas are tissue adhesion areas. It is analyzed and shown through scanning electron microscope (SEM) that non-uniform areas are tissue adhesion positions. From the results, obvious tissue adhesion phenomena occur after the medical appliances in Groups A and B are implanted under mouse skin. Tissue adhesion area on the surface of the medical appliance in Group C is apparently fewer than those for Group A and B. In other words, the surface treatment method according to the present invention can improve the ratio of tissue anti-adhesion, thereby improving the effect of tissue anti-adhesion of the products.

### Second Embodiment

For the metal implant (that is, the medical appliance), in the second embodiment, titanium metal is taken as an example. A thickness of a native oxide layer off its surface is about 10 nm. An electrochemical anodization process is performed on the titanium metal. Conditions for the electrochemical anodization process include a voltage of 55 V and process duration of 3 minutes. The result shows that the color of the surface of the titanium metal is golden after the electrochemical anodization process. The thickness of the oxide layer is greater than 100 nm. Subsequently, the thickness of the functional polymer 16 grafted through the electrodeposition method is greater than 80 nm. Therefore, through the electrochemical anodization process, the thickness of the oxide layer on the surface of the titanium metal can also be increased, so as to increase the amount of hydroxyl groups on the surface effectively, thereby improving the amount of grafted special functional polymer 16. The functional polymer 16 is transparent, and thus the color of the medical appliance is also golden.

### Third Embodiment

For the metal implant (that is, the medical appliance), in the third embodiment, titanium metal is taken as an example. A thickness of the native oxide layer off the surface is about 10 nm. A heating process is performed on the titanium metal. Conditions of the heating process include the following steps. A temperature 600° C. is provided in a uniformly heated space far one hour. It is analyzed the surface of the titanium metal is interlaced gray-blue-green after the heating process. The thickness of the oxide layer is greater than 1 μm. Subsequently, the thickness of the functional polymer 16 grafted through the electrodeposition method is greater than 150 nm. Therefore, through the heating process, the thickness of the oxide layer on the surface of the titanium metal can also be increased, so as to increase the amount of hydroxyl groups on the surface effectively, thereby improving an amount of grafted special functional polymer 16. The functional polymer 16 is transparent and therefore the color of the medical appliance is also interlaced gray-blue-green.

In conclusion, in the present invention, the intermediate layer is formed in different modes. Different colors are presented according to different thickness of the intermediate layer. Therefore, the products are obviously recognizable. In addition, due to the formation of the intermediate layer, the number of the hydroxyl groups (—OH) on the surface of the metal layer is increased.

Additionally, in the present invention, the special functional polymer is then electrochemically grafted on the hydroxyl groups (—OH) on the surface of the metal layer. As the method for forming the intermediate layer increases the number of the hydroxyl groups on the surface effectively, the amount of the grafted special functional polymer is increased, so that the tissue anti-adhesion effects of the product is improved.

Although the present invention is disclosed above with reference to the above embodiments, the embodiments are not intended to limit the present invention. Equivalent replacements of variations and modifications made by any person skilled in the art without departing from the spirit and scope of the present invention still fall with the protection scope of the present invention.

What is claimed is:

1. A surface treatment method for a medical appliance, comprising:
providing a metal layer;
forming an intermediate layer on a surface of the metal layer, wherein a thickness of the intermediate layer is greater than a thickness of a native oxide layer of the surface of the metal layer; and
grafting a dissoluble functional polymer on the intermediate layer through an electrodeposition process.

2. The surface treatment method according to claim 1, wherein the metal layer is a titanium metal, an alloy containing titanium element, a vitallium, or a stainless steel.

3. The surface treatment method according to claim 2, wherein the intermediate layer is a titanium oxide layer.

4. The surface treatment method according to claim 1, wherein the electrodeposition process comprises:
placing the metal layer in an electrolyte fluid containing the functional polymer; and
providing a current to perform the electrodeposition process.

5. The surface treatment method according to claim 1, wherein the functional polymer is a polymer having a terminal amino group.

6. The surface treatment method according to claim 5, wherein the polymer having the terminal amino group is poly ethylene glycol (PEG).

7. The surface treatment method according to claim 1, wherein the intermediate layer is formed through an electrochemical anodization process and the electrochemical anodization process comprises:
placing the metal layer at an anode and placing an electrode at a cathode; and
providing a current at the metal layer.

8. The surface treatment method according to claim 7, wherein a reaction temperature of the electrochemical anodization process is in a range between −20° C. and 60° C. and an externally applied voltage of the anodization process is between 5 volts (V) and 200 V.

9. The surface treatment method according to claim 8, wherein the reaction temperature of the electrochemical anodization process is preferably in a range between −5° C. and 35° C. and the externally applied voltage of the anodization process is preferably between 40 V and 60 V.

10. The surface treatment method according to claim 1, wherein the intermediate layer is formed through a chemical soak process, and the chemical soak process comprises soaking the metal layer in an acid solution, and the acid solution is a solution containing hydrogen peroxide (H₂O₂).

11. The surface treatment method according to claim 1, wherein the intermediate layer is formed through a heating process, and the heating process comprises placing the metal layer in a uniformly heated space and providing a suitable temperature in a given time duration.

12. A medical appliance, comprising:
a metal layer;
an intermediate layer, formed on a surface of the metal layer, wherein a thickness of the intermediate layer is greater than a thickness of a native oxide layer of the surface of the metal layer surface; and
a dissoluble functional polymer, grafted on the intermediate layer.

13. The medical appliance according to claim 12, wherein the metal layer is a titanium metal, an alloy containing titanium element, a vitallium, or a stainless steel.

14. The medical appliance according to claim 13, wherein the intermediate layer is a titanium oxide layer.

15. The medical appliance according to claim 14, wherein a color of a medical appliance comprising the intermediate layer is different from a color of a medical appliance comprising a native oxide layer.

16. The medical appliance according to claim 15, wherein the color of the medical appliance is golden, grayish blue, or interlaced gray-blue-green.

17. The medical appliance according to claim 14, wherein the thickness of the intermediate layer is between 10 nanometers (nm) and 20 micrometers (μm).

18. The medical appliance according to claim 12, wherein the medical appliance is an object implanted in bodies, and the object is a bone screw, a bone plate, or a cardiac stent.

19. The medical appliance according to claim 12, wherein the medical appliance is an object inserting in bodies temporarily, and the object is a metal guide wire or a surgical instrument.

20. The medical appliance according to claim 12, wherein the medical appliance is an object operated outside bodies, and the object is a surgical wrench or a medical metal basin.
